(19) United States
(12) Patent Application Publication
(10) Pub. No.: US 2018/0263873 A1
(43) Pub. Date: Sep. 20, 2018

(54) AGENTS FOR REDUCTIVE DECOLORIZATION OF DYED KERATINOUS FIBERS

(71) Applicant: Henkel AG & Co. KGaA, Duesseldorf (DE)

(72) Inventors: Juergen Schoepgens, Schwalmtal (DE); Torsten Lechner, Langenfeld (DE)

(73) Assignee: Henkel AG & Co. KGaA, Duesseldorf (DE)

(21) Appl. No.: 15/920,482

(22) Filed: Mar. 14, 2018

(30) Foreign Application Priority Data
Mar. 15, 2017 (DE) 102017204283.4

(51) Int. Cl. A61K 8/22; A61K 8/44; A61K 8/46; A61Q 5/08

(52) U.S. Cl. CPC A61K 8/22; A61K 8/44; A61K 2800/882; A61Q 5/08; A61K 8/46

(57) ABSTRACT

Multi-component package units (kits-of-parts), ready-to-use agents for reductive decolorizing of dyed keratinous fibers, and methods for reductive decolorizing of dyed keratinous fibers are provided herein. In an embodiment, a multi-component package unit (kit-of-parts) for reductive decolorizing of keratin fibers includes, separately packaged, a first container (A) and a second container (B). The first container (A) includes a cosmetic agent (a) and the second container (B) includes a cosmetic agent (b). Agent (a) in the first container (A) includes (a1) thiourea. Agent (b) in container (B) includes (b1) one or multiple oxidizing agents chosen from the group of hydrogen peroxide, potassium persulfate, sodium persulfate and/or ammonium persulfate.

### CROSS-REFERENCE TO RELATED APPLICATION

This application claims priority to German Patent Application No. 10 2017 204 283.4, filed Mar. 15, 2017, which is incorporated herein by reference in its entirety.

### TECHNICAL FIELD

The present disclosure relates to the cosmetic sector. The subject of the present disclosure is a multi-component package unit (kit-of-parts or kit) for reductive decolorization of dyed keratinous fibers which includes two separately packaged containers (A) and (B). Container (A) contains a first cosmetic agent (a) with thiourea. Container (B) contains a second cosmetic agent (b), which is exemplified by its content of one or multiple oxidants.

### BACKGROUND

An additional subject of the present disclosure is a ready-to-use agent for reductive decolorization of dyed keratinous fibers which is obtained by mixing the two agents (a) and (b) described above.

A third subject of the present disclosure is a method for reductive decolorization of dyed keratinous fibers, with which the multi-component package unit and/or ready-to-use decolorizing agent described above is used.

Preparations for tinting and coloring hair are an important type of cosmetic agent. They can serve to tint the natural hair color to a lesser or greater degree depending on the preferences of each and every person, achieve a completely different hair color or cover unwanted color shades, such as shades of gray, for example. Routine hair dyes are formulated either on the basis of oxidation dyes or on the basis of partially-oxidizing dyes, depending on the preferred color and/or permanency of the dye. Combinations of oxidizing dyes and partially-oxidizing dyes are frequently used to obtain special nuances.

Dyes formulated on the basis of oxidation dyes lead to brilliant and permanent color shades. However, they do require the use of strong oxidants, such as hydrogen peroxide solutions, for example. Said dyes contain oxidative dye precursors, so-called developer components and coupler components. The developer components join together or couple with one or more coupler components to form, under the influence of oxidants or atmospheric oxygen, the actual colorants per se.

Dyes formulated on the basis of partially-oxidizing dyes are often used to achieve temporary colors. Partially-oxidizing dyes are dye molecules that coat the hair itself and do not require an oxidative process to create the color. Important representatives of this dye class include triphenylmethane dyes, azo dyes, anthraquinone dyes or nitrobenzene dyes, each of which can carry cationic or anionic groups.

With all said dyeing methods, however, the color may need to be reversed, either whole or in part, for various reasons. A partial removal of the color may be the ideal solution, for example, if the color result has a darker effect on the fibers than desired. On the other hand, a complete removal of the color may be desired in some cases. It is conceivable, for example, that the hair is to be colored or tinted in a particular way for a specific occasion, and the original color is to be restored after a few days.

Technical literature also discloses decolorizing agents and methods. The oxidative decolorization of dyed hair, by employing a routine blonding agent for example, is a well-known method from the prior art. With this process, however, the fibers can also be damaged through the use of strong oxidants.

Moreover, reductive processes for decolorization have already been described. European Patent Application EP 1300136 A2 discloses, for example, a method for hair treatment, wherein the hair is colored in a first step and then reductively decolorized again in a second step. Said reductive decolorization is achieved by employing a formulation containing a diothine salt and a surfactant. In WO 2008/055756 A2, the reductive decolorization of keratin fibers is achieved using a mixture formed from a reducing agent and an absorption agent.

When reductive decolorizing agents are used, the decolorization effect is achieved by reducing the dyes located on the keratin fibers and/or hair. The reduction process usually involves converting the dyes to their reduced forms and/or leuco forms. This method involves reducing the double bonds present in the dyes, thus interrupting the chromophoric system of the dyes and converting the dye into a colorless form.

Normally, strong reducing agents must be used for the reduction of dyes. These reducing agents are very reactive compounds that are often unstable in a hydrous solution and—depending on the pH value of the solution—decompose to a greater or lesser degree. For example, the reductive decolorizing agent sodium dithionite known from the prior art is sensitive to atmospheric oxygen and decomposes slowly in a hydrous solution. By increasing the pH value, this decomposition reaction can be delayed. The adjustment to a slightly alkali pH value stabilizes hydrous dithionite solutions so that the solution can be stored for several weeks to months with the absence of oxygen. However, if the reductive decolorizing agent should no longer be stored and under storage conditions with high temperatures, packaging in a solution, particularly a hydrous solution is not the method of choice.

Other reducing agents, such as formadine sulfinic acid are not stable in hydrous solutions for an extended time so that a suitable method for providing the reducing agent in a storable form is still sought.

### BRIEF SUMMARY

Multi-component package units (kits-of-parts), ready-to-use agents for reductive decolorizing of dyed keratinous fibers, and methods for reductive decolorizing of dyed keratinous fibers are provided herein. In an embodiment, a multi-component package unit (kit-of-parts) for reductive decolorizing of keratin fibers includes, separately packaged, a first container (A) and a second container (B). The first container (A) includes a cosmetic agent (a) and the second container (B) includes a cosmetic agent (b). Agent (a) in the first container (A) includes (a1) thiourea. Agent (b) in container (B) includes (b1) one or multiple oxidizing agents chosen from the group of hydrogen peroxide, potassium persulfate, sodium persulfate and/or ammonium persulfate.

In another embodiment, a ready-to-use agent for reductive decolorizing of dyed keratinous fibers includes (a1) thiourea and (b1) one or multiple oxidizing agents chosen from the group of hydrogen peroxide, potassium persulfate, sodium persulfate and/or ammonium persulfate.

In another embodiment, a method for reductive decolorizing of dyed keratinous fibers includes the following steps in the specified sequence. A first agent (a) is mixed with a second agent (b), where agent (a) includes (a1) thiourea, and agent (b) includes (b1) one or multiple oxidizing agents chosen from the group of hydrogen peroxide, potassium persulfate, sodium persulfate and/or ammonium persulfate. The mixture of (a) and (b) is mixed with a third agent (c), where agent (c) includes a cosmetic agent (c). The mixture of (a), (b), and (c) is applied on the dyed keratinous fibers and the decolorizing agent is allowed to take effect. The decolorizing agent is rinsed off of the keratinous fibers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure addresses the problem of providing packaging possibilities, agents and processes which enable comfortable, consistent and quick production of a ready-to-use decolorizing agent. The ready-to-use decolorizing agent produced in this manner should decolorize the keratinous fibers as consistently and effectively as possible. Furthermore, the decolorizing agent is exemplified by high storage stability and still has a high decolorizing effect after a long storage time at high temperatures.

In the context of the work leading up to this present disclosure, a method was found that enables in situ production of suitable reducing agents for reductive color removal shortly before us. The intermediate production of the reducing agent is enabled by mixing two agents that are provided in the form of a special multi-component package unit (kit-of-parts). This multi-component package unit includes two separately packaged containers (A) and (B), where containers (A) and (B) contain cosmetic agents (a) and (b) respectively. Agent (A) contains thiourea. Agent (b) is a cosmetic carrier which contains at least one oxidant, particularly hydrogen peroxide.

To produce the ready-for-use agent decolorizing agent, agents (a) and (b) are mixed together. The thiourea previously packaged separately comes into contact with the oxidant, hydrogen peroxide, in particular. When the two substances come into contact with each other they react and intermediately form the reducing agent formamidine sulfinic acid. It has been found that a mixture develops already after a short reaction time of from about 10 to about 30 minutes, which is very well-suited for decolorizing dyed keratinous fibers. Since the formamidine sulfinic acid is not produced until shortly before application of the decolorizing agent, all stability problems that occur when formamidine sulfinic acid must be stored for an extended time can be avoided.

A first subject of the present disclosure, is a multi-component package unit (kit-of-parts) for reductive decolorizing of keratin fibers, comprising

(I) a first container (A) containing a cosmetic agent (a) and
(II) a second container (B) containing a cosmetic agent (b),
wherein
agent (a) in the first container (A) contains
   (a1) thiourea and
agent (b) in container (B) contains
   (b1) one or multiple oxidizing agents from the group including of hydrogen peroxide, potassium persulfate, sodium persulfate and/or ammonium persulfate.

Keratinous fibers, keratin-containing fibers or keratin fibers are furs, wool, feathers and, in particular, human hair. Although the agents as contemplated herein are most suitable for lightening and coloring keratinous fibers and/or human hair, they can in principle be used for other purposes.

The expression “dyed keratinous fibers” means keratinous fibers, which were dyed by employing conventional cosmetic dyes known to a person skilled in the art. The expression “dyed keratinous fibers” means in particular hair that have been dyed by employing oxidative dyes and/or partially oxidizing dyes known from the prior art. In this context, explicit reference is made to the known monographies, e.g. Kh. Schrader, Grundlagen and Rezepturen der Kosmetika [Cosmetic principles and formulas], 2nd Edition, Hüthig Buch Verlag, Heidelberg, 1989, which reflect the corresponding knowledge of a person skilled in the art.

Agent (a) in Container (A)

The inventive multi-component package unit (kit-of-parts) comprises a first separately packaged container (A) with a cosmetic agent (a). Agent (a) is exemplified in that it contains thiourea as an ingredient.

Thiourea has structural formula (I), the CAS number 62-56-6 and is commercially available from various suppliers, such as Sigma Aldrich.

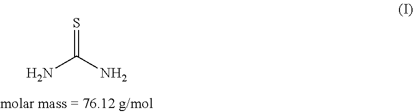

Thiourea is preferably used in specific quantity ranges in agent (a). Agent (a) preferably contains thiourea in an amount of from about 1.0 to about 80.0 wt. %, from about 3.0 to about 60.0 wt. %, more preferably from about 5.0 to about 40.0 wt. %, particularly from about 7.0 to about 20.0 wt. %. These specifications in percent by weight are relative to the total weight of agent (a).

Therefore, preference is given to a multi-component package unit (kit-of-parts) for reductive decolorization of dyed keratinous fibers exemplified in that the agent (a) in the first container (A) contains—relative to the total weight of agent (a)—from about 1.0 to about 80.0 wt. %, preferably from about 3.0 to about 60.0 wt. %, more preferably from about 5.0 to about 40.0 wt. % and particularly from about 7.0 to about 20.0 wt. % thiourea.

Agent (a) can contain thiourea in a hydrous carrier. This can, for example, be a suitable hydrous or hydrous-alcoholic carrier. Carriers such as creams, emulsions, gels or surfactant-containing, foaming solutions, such as shampoos, foaming aerosols, foam formulations or other preparations suitable for application on the hair, are used for the purpose of reductive decolorization. Agents for the reductive decolorization of keratinous fibers are preferably creams, emulsions or free-flowing gels.

The optimal pH range for reaction of thiourea (a1) with the oxidant (b1) (hydrogen peroxide, in particular) is from about 2.0 to about 6.5. In order to achieve the quickest, most effective and most complete reaction of reactants (a1) and (b1), therefore, it is particularly advantageous that the mixture of agent (a) and (b) has a pH value in the range of from about 2 to about 6.5, particularly from about 3.0 to about 5.5. Alkalizing agents and/or acidifying agents are preferably used to adjust this pH value.

Agent (b) preferably contains the oxidant or oxidants (b1) in the form of a solution. Solutions of hydrogen peroxide adjusted to a pH value below about 3, in particular, are used for stability purposes. Therefore, if the mixture of agents (a) and (b) has a pH value closer to the range of from about 4 to about 6, it is advantageous if an alkalizing agent used to adjust the optimal pH value range is incorporated into agent (a) or packaged separately in a third agent (c).

The pH value can be measured by employing a gas electrode, for example, which is usually commercially available in the form of a combination electrode. Before the pH value is measured, the gas electrodes are usually calibrated with calibration solutions of a known pH value. The pH values as defined by the present disclosure are pH values that were measured at a temperature of 22° C.

The desired pH value can be set by employing various alkalizing agents. Suitable alkalizing agents as contemplated herein are selected from the group formed from ammonia, alkanolamines, alkali metal hydroxides, alkali metal metasilicates, alkalimetal phosphates and alkali metal hydrogen phosphates. Preferred inorganic alkalizing agents are sodium hydroxide, sodium hydroxide, sodium silicate and sodium metasilicate. Organic alkalizing agents usable as contemplated herein are preferably selected from monoethanolamine, 2-amino-2-methylpropanol and triethanolamine. The basic amino acids that can likewise be used as alkalizing agents are preferably can be selected from the group formed of arginine, lysine, ornithine, and histidine.

Therefore, preference is given to a multi-component package unit (kit-of-parts) for reductive decolorization of dyed keratinous fibers exemplified in that
agent (a) in the first container (A) contains
   (a2) one or more alkalizing agents from the group including ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate.

To finely adjust the pH value, small amounts of acidifying agents can also be used in agent (a). Acidification agents suitable as contemplated herein include citric acid, lactic acid, tartaric acid, malic acid, acetic acid, dipicolinic acid, etidronic acid or also dilute mineral acids (such as hydrochloric acid, sulphuric acid, phosphoric acid).

In a further embodiment, it can also be necessary that agent (a) is essentially packaged anhydrously. In this context, the term “essential anhydrous” is understood to mean that the water content of agent (a) is 10.0 wt. % at the most. Specific amounts of water can, for example, be introduced to the agent if a raw material in the form of a hydrate or a solution is used. The water content of agent (a), however, is preferably below about 10.0 wt. %, more preferably below about 5.0 wt. %, more preferably below about 2.5%, particularly below about 0.1 wt. %. In the process, all specifications in percent by weight are relative to the total weight of agent (a).

Therefore, particular preference is given to a multi-component package unit (kit-of-parts) for reductive decolorization of dyed keratinous fibers exemplified in that the agent (a) in the first container (A) contains—relative to the total weight of agent (a)—water content below about 10.0 wt. %, preferably below about 5.0 wt. %, more preferably below about 2.5 wt. % and particularly below about 0.1 wt. %.

The essentially anhydrous agent (a) can, for example, be a powder or a paste. If agent (a) is used in the form of a powder, dust formation must be avoided and/or the powder must be dedusted. Therefore, it is particularly beneficial if agent (a) is provided in the form of a paste.

In order to obtain a pasty agent (a), the thiourea (a1) can be incorporated into a fat-containing carrier. In the process, the consistency of the past is co-determined by the melting point of the fat components.

Therefore, preference is given to a multi-component package unit (kit-of-parts) for reductive decolorization of dyed keratinous fibers exemplified in that the agent (a) in the first container (A) contains

(a3) one or multiple fatty constituents (a2) from the group including C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and C₁₂-C₃₀ fatty acid esters containing hydrocarbons and/or silicone oils.

It is particularly preferred that agent (a) contains one or multiple fat components from the group including C₁₂-C₃₀ fatty alcohols C₁₂-C₃₀ fatty acid triglycerides, C₁₂-C₃₀-fatty acid monoglycerides, C₁₂-C₃₀ fatty acid diglycerides, C₁₂-C₃₀ fatty acid esters and/or hydrocarbons.

To the extent required by the present disclosure, “fatty constituents” are organic compounds with a water solubility at room temperature (22° C.) and atmospheric pressure (760 mm Hg) of less than about 1 wt. %, preferably less than about 0.1 wt. %.

The definition of fatty constituents explicitly includes only uncharged (i.e. nonionic) compounds. Fatty constituents have at least one saturated or unsaturated alkyl group with at least 12 C-atoms. The molar weight of the fatty constituents is a maximum about 5000 g/mol, preferably maximum about 2500 g/mol and even more preferably a maximum of about 1000 g/mol. The fatty constituents are neither polyoxyalkylated nor polyglycerylated compounds. In this connection, polyalkoxylated compounds are such compounds for which 2 aklylene oxide units were implemented in the production thereof. Analogously, polyglycerated compounds are such compounds for which two glycerin units were implemented in the production thereof.

Since only nonionic substances are considered fatty constituents within the context of the present disclosure, charged components, such as fatty acids and salts thereof do not fall under the group including fatty constituents.

Preferred fatty constituents are the constituents from the group including C₁₂-C₃₀ fatty alcohols C₁₂-C₃₀ fatty acid triglycerides, C₁₂-C₃₀-fatty acid monoglycerides, C₁₂-C₃₀ fatty acid diglycerides, C₁₂-C₃₀ fatty acid esters and hydrocarbons.

The C₁₂-C₃₀ fatty alcohols can be saturated, one or more unsaturated, linear or branched fatty alcohols with from about 12 to about 30 C-atoms.

Examples of preferred linear, saturated C₁₂-C₃₀ fatty alcohols are dodecan-1-ol (dodecylalcohol, laurylalcohol), tetradecan-1-ol (retradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Preferred linear, unsaturated fatty alcohols are (9Z)-octadec-9-en-1-ol (oleyl alcohol), (9E)-octadec-9-en-1-ol (elaidyl alcohol), (9Z,12Z)-cctadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidyl alcohol ((5Z,8Z,11Z,14Z)-eicosa-5,8,11,14-tetraen-1-ol), erucyl alcohol ((13Z)-docos-13-en-1-ol) and/or brassidyl alcohol ((13E)-docosen-1-ol).

The preferred typical branched fatty alcohols are 2-octyl-dodecanol, 2-hexyl-dodecanol and/or 2-butyl-dodecanol.

To the extent required by the present disclosure, a C₁₂-C₃₀ fatty acid triglyceride is the triester of the trivalent alcohol glycerine with three equivalent fatty acids. Both identically structured and different fatty acids within a triglyceride molecule can be involved in the ester formation.

To the extent required by the present disclosure, fatty acids are saturated or unsaturated, unbranched or branched, unsubstituted or substituted Cᵤ-Cₘ carboxylic acids. Unsaturated fatty acids can be unsaturated or polyunsaturated. The C—C double bond(s) of an unsaturated fatty acid can have the cis- or trans configuration.

Fatty acid diglycerides are exemplified by their particular suitability, for which at least one of the ester groups, based on glycerine, is formed with a fatty acid, which is selected from dodecan acid (laurin acid), tetradecan acid (myristine acid), hexadecanoic acid (palmitic acid), tetracosanic acid (lignoceric acid), octadecanoic acid (stearic acid), eicosanoic acid (arachidic acid), docosanic acid (behenic acid), petroselinic acid [(Z)-6-octadecenoic acid], palmitoleic acid [(9Z)-hexadec-9-enic acid], oleic acid [(9Z)-octadec-9-enic acid], elaidinoic acid [(9E)-octadec-9-enic acid], erucic acid [(13Z)-docos-13-enic acid], linoleic acid [(9Z,12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] and/or nervonic acid [(15Z)-tetracos-15-enic acid].

The fatty acid triglycerides can also be from natural sources. The fatty acid triglycerides occurring in soy bean oil, peanut oil, sunflower oil, macadamia nut oil, drumstick tree oil, apricot kernel oil, manila oil and/or possibly hardened castor oil, and the mixtures thereof are particularly suitable for use in agent (a) as contemplated herein.

A C₁₂-C₃₀ fatty acid monoglyceride is the monoester of the trivalent alcohol glycerine with an equivalent fatty acid. Either the middle hydroxy group including the glycerine or the final hydroxy group including the glycerin can be esterified with the fatty acid.

The C₁₂-C₃₀ fatty acid triglycerides are exemplified by their particular suitability, for which at least one hydroxy group of the glycerine is esterified, wherein the fatty acids are selected from dodecan acid (laurin acid), tetradecan acid (myristine acid), hexadecanoic acid (palmitic acid), tetracosanic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enic acid], oleic acid [(9Z)-octadec-9-enic acid], elaidinoic acid [(9E)-octadec-9-enic acid], erucic acid [(13Z)-docos-13-enic acid], linoleic acid [(9Z,12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] or nervonic acid [(15Z)-tetracos-15-enic acid].

A C₁₂-C₃₀ fatty acid diglyceride is the diester of the trivalent alcohol glycerine with two equivalent fatty acids. Either the middle and/or an independent hydroxy group of the glycerine with two equivalent fatty can be esterified with two equivalent fatty acids or both final hydroxy groups of the glycerin are each esterified with one fatty acid. The glycerin can be esterified with two identically structured or two different fatty acids.

Fatty acid diglycerides are exemplified by their particular suitability, for which at least one of the ester groups, based on glycerine, is formed with a fatty acid, which is selected from dodecan acid (laurin acid), tetradecan acid (myristine acid), hexadecanoic acid (palmitic acid), tetracosanic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enic acid], oleic acid [(9Z)-octadec-9-enic acid], elaidinoic acid [(9E)-octadec-9-enic acid], erucic acid [(13Z)-docos-13-enic acid], linoleic acid [(9Z,12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] and/or nervonic acid [(15Z)-tetracos-15-enic acid].

In the context of the present disclosure, a C₁₂-C₃₀ fatty acid ester is understood to mean the monoester from a fatty acid and an aliphatic monovalent alcohol, where the alcohol comprises up to 6 carbon atoms. Suitable alcohols include, for example, ethanol, n-propanol, isopropanol, 1-butanol, isobutanol, tert-butanol, n-pentanol, iso-pentanol or n-hexanol. Ethanol and isopropanol are preferred alcohols.

Preferred C₁₂-C₃₀ fatty acid esters are the esters with which esterification of the alcohols and/or isopropanol are formed with one of the fatty acids from the group of dodecan acid (lauric acid), tetradecan acid (myristine acid), hexadecanoic acid (palmitic acid), tetracosanic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enic acid], oleic acid [(9Z)-octadec-9-enic acid], elaidinoic acid [(9E)-octadec-9-enic acid], erucic acid [(13Z)-docos-13-enic acid], linoleic acid [(9Z,12Z)-octadeca-9,12-dienic acid, linoleic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-Icosa-5,8,11,14-trienoic acid] and/or nervonic acid [(15Z)-tetracos-15-enic acid]. Particular preference is given to isopropyl myristate as a fatty acid ester.

Hydrocarbons are exclusively compounds including exclusively the atoms hydrocarbon and hydrogen in compounds with from about 8 to about 250 carbon atoms, preferably about 150 carbon atoms. In this context, aliphatic hydrocarbons such as mineral oils, liquid paraffin oils (e.g. paraffinum liquidum or paraffinum perliquidum), isoparaffin oils, semi-solid paraffin oils, paraffin waxes, hard paraffin (paraffinum solidum), vaseline and polydecene are preferred.

In this context, liquid paraffin oils (paraffinum liquidum and paraffinum perliquidum) have proven to be particularly suitable. The most preferable hydrocarbon is paraffinum liquidum, also referred to as white oil. Paraffinum liquidum is a mixture of cleaned, saturated, aliphatic hydrocarbons, which mainly includes hydrogen chains with a C-chain distribution from about 25 to about 35 C-atoms.

The fatty constituents can be the cosmetic carrier of agent (a) and also—depending on the nature and amount of the fat which is used—have a great influence on the consistency of the agent. In this context, it has been found to be particularly preferable that agent (a) has one or multiple fatty constituents in a total amount of from about 10 to about 90 wt. %, preferably from about 20 to about 60 wt. % and particularly from about 25 to about 50 wt. %, where these quantity specifications are relative to total weight of the agent (a).

Agent (b) in Container (B)

The second cosmetic agent (b) is contained in the second container (B) of the inventive multi-component package unit. This agent is exemplified in that it contains one or multiple oxidants (b1) from the group of hydrogen peroxide, potassium persulfate, sodium persulfate and/or ammonium persulfate.

Agent (b) is preferably hydrous or packaged in a hydrous manner. Cosmetic agent (b) can be, for example, an agent with a suitable hydrous or hydrous-alcoholic carrier. Carriers such as creams, emulsions, gels or surfactant-containing, foaming solutions, such as shampoos, foaming aerosols, foam formulations or other preparations suitable for application on the hair, are used for the purpose of reductive decolorization. Agents for the reductive decolorization of keratinous fibers are preferably creams, emulsions or free-flowing gels. It is particularly preferred that agent (b) is formulated as an emulsion.

It is particularly preferred that hydrogen peroxide is used as oxidant (b1) in agent (b). Hydrogen peroxide is especially well-suited for use in the form of a hydrous solution. Hydrogen peroxide also reacts particularly well with thiourea and delivers the formamidine sulfinic acid desired as a reaction product in a particularly defined reaction with a high yield.

Therefore, particular preference is given to a multi-component package unit (kit-of-parts) for reductive decolorization of dyed keratinous fibers exemplified in that the agent (b) in the second container (B) contains

(b1) hydrogen peroxide.

Furthermore, preference is given to a multi-component package unit (kit-of-parts) for reductive decolorizing of keratin fibers, comprising

(I) a first container (A) containing a cosmetic agent (a) and
(II) a second container (B) containing a cosmetic agent (b),
wherein
agent (a) in the first container (A) contains
   (a1) thiourea and
   (a2) one or more alkalizing agents from the group of ammonia, 2-aminoethan-1-ol, 2-amino-2-methylpropane-1-ol, arginine, lysine, ornithine, histidine and
agent (b) in container (B) contains
   (b1) hydrogen peroxide.

Furthermore, preference is given to a multi-component package unit (kit-of-parts) for reductive decolorizing of keratin fibers, comprising

(I) a first container (A) containing a cosmetic agent (a) and
(II) a second container (B) containing a cosmetic agent (b),
wherein
agent (a) in the first container (A) contains
   (a1) thiourea and
   (a2) one or multiple alkalizing agents from the group of potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate and
agent (b) in container (B) contains
   (b1) hydrogen peroxide.

The oxidant or oxidants (b1) are preferably used in specific quantity ranges in agent (b). Agent (b) in container (B) preferably contains—relative to the total weight of agent (b)—one or multiple oxidants (b1) in a total amount of from about 0.1 to about 12.0 wt. %, preferably from about 1.0 to about 10.0 wt. %, more preferably from about 3.0 to about 9.0 wt. % and particularly from about 4.0 to about 8.0 wt. %.

It is particularly preferred that agent (b) in container (B) preferably contains —relative to the total weight of agent (b)—one or multiple oxidants (b1) in a total amount of from about 0.1 to about 12.0 wt. %, preferably from about 1.0 to about 10.0 wt. %, more preferably from about 3.0 to about 9.0 wt. % and particularly from about 4.0 to about 8.0 wt. %.

It is even more preferable that agent (b) in container (B) contains from about 0.1 to about 12.0 wt. %, preferably from about 1.0 to about 10.0 wt. %, more preferably from about 3.0 to about 9.0 wt. % and particularly from about 4.0 to about 8.0 wt. % hydrogen peroxide relative to the total weight of agent (b).

In another particularly preferred embodiment, an inventive multi-component package unit (kit-of-parts) is exemplified in that agent (b) in container (B) contains from about 0.1 to about 12.0 wt. %, preferably from about 1.0 to about 10.0 wt. %, more preferably from about 3.0 to about 9.0 wt. % and particularly from about 4.0 to about 8.0 wt. % hydrogen peroxide relative to the total weight of agent (b).

Agent (b) contains oxidants, with particular preference being given to hydrogen peroxide. Hydrogen peroxide can be used in the form of its addition products, particularly in the form of a hydrous solution. To stabilize the hydrous solutions, agents (b) are preferably adjusted to an acidic pH value from about 2 to about 7, preferably from about 2 to about 5, particularly from about 2 to about 3.

To adjust the pH value, it has been found that one or multiple acids from the group of citric acid, tartaric acid, malic acid, lactic acid, acetic acid, sulphuric acid, hydrochloric acid, phosphoric acid, methane sulfonic acid, benzoic acid, malonic acid, oxalic acid, oxalocetic acid and/or 1-hydroxyethane-1,1-diphosphonic acid are suitable. Preferably, the acid or acids are selected from the group of citric acid, tartaric acid, malic acid, lactic acid, methanesulfonic acid, oxalic acid, malonic acid, benzoic acid, hydrochloric acid, sulphuric acid, phosphoric acid and/or 1-hydroxyethane-1,1-diphosphonic acid.

Furthermore, particular preference is given to a multi-component package unit (kit-of-parts) for reductive decolorizing of keratin fibers, which is separately packaged, comprising

(I) a first container (A) containing a cosmetic agent (a) and
(II) a second container (B) containing a cosmetic agent (b),
wherein
agent (a) in the first container (A) contains
   (a1) thiourea and
   (a2) one or multiple alkalizing agents and
agent (b) in container (B) contains
   (b1) hydrogen peroxide and
   (b2) one or multiple alkalizing agents,

Furthermore, particular preference is given to a multi-component package unit (kit-of-parts) for reductive decolorizing of keratin fibers, which is separately packaged, comprising

(I) a first container (A) containing a cosmetic agent (a) and
(II) a second container (B) containing a cosmetic agent (b),
wherein
agent (a) in the first container (A) contains
   (a1) thiourea and
   (a2) one or more alkalizing agents from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate, and
agent (b) in container (B) contains
   (b1) hydrogen peroxide and
   (b2) one or multiple acids from the group of 1-hydroxyethane-1,1-diphosphonic acid, citric acid, tartaric acid, malic acid, lactic acid, methanesulfonic acid, oxalic acid, malonic acid, benzoic acid, hydrochloric acid, sulfuric acid and/or phosphoric acid.

As described above, agent (b) in container (B) is preferably a hydrous cosmetic carrier formulation. The water content of this formulation is from about 30 to about 97 wt. %, preferably from about 40 to about 95 wt. %, more preferably from about 50 to about 93 wt. % and particularly from about 60 to about 91 wt. %. relative to the total weight of agent (b).

Therefore, particular preference is given to a multi-component package unit (kit-of-parts) for reductive decolorization of dyed keratinous fibers exemplified in that agent (b) contains from about 30 to about 97 wt. %, preferably from about 40 to about 95 wt. %, more preferably from about 50 to about 93 wt. % and particularly from about 60 to about 91 wt. % water relative to the total weight of agent (b).

Mixture of Agent (a) Plus (b)

To produce the ready-to-use decolorizing agent, agents (a) and (b) are mixed together, where the total amount of agent (a) in container (A) is mixed with the total amount of agent (b) in container (B).

The mixture can take place, for instance, by transferring the entire contents of container (A) to container (B) (in this case, container (B) is larger than container (A)). The present disclosure also allows for the transfer of the entire contents of container (B) to container (A) (in this case, container (A) is larger to the appropriate extent).

Thiourea (a1) and (oxidants (b1) react with each other in the mixture of agents (a) and (b). The extent and speed of this reaction can be controlled with the molar ratio in which the two reactants (a1) and (b1) are used in relation to each other.

It has been found that a complete reaction of the thiourea (a1) can be achieved, in particular, when the oxidants (b1) are used in at least an equimolar amount, but preferably in an excess of two to five times the amount of thiourea (a1).

Therefore, the oxidants (b1) are used in a molar excess of 1 to 5 times in comparison with the thiourea (a1) in the total amount of agents (a) plus (b). This guarantees that the thiourea (a1) reacts as completely as possible.

Since the progress of the reaction depends on the amounts of thiourea (a1) and oxidizing agent(s) (b1) in the mixture of agents (a) plus (b), all specifications relating to the weight ratio (b1)/(a1) are also relative to the total weight of agents (a) plus (b).

### Example 1

Container (A) contains 20 g of agent (a). Agent (a) contains (a1) 8.0 g of thiourea.

The molar mass of thiourea is 76.12 g/mol.
Agent (a) (20 g) contains 0.105 mol of thiourea.
Container (B) contains 80 g of agent (b). Agent (b) contains (b1) 7.2 g of hydrogen peroxide. The molar mass of hydrogen peroxide is 34.02 g/mol.
Agent (b) (80 g) contains 0.211 mol of hydrogen peroxide.
To produce the ready-to-use decolorizing agent, 20 g of agent (a) is mixed together with 80 g of agent (b) (total weight of agent (a) plus (b)=100 g).
The total amount of agents (a)+(b) includes:
(a1) 8.0 g thiourea (0.105 mol in agent (a) plus (b))
(b1) 7.2 g hydrogen peroxide (0.211 mol in agent (a) plus (b))
The molar ratio of the total amount of all oxidants (b1) contained in agent (b) to the total weight of agents (a) plus (b) is 0.211 mol/0.105 mol=2.0 relative to the total weight of agents (a) plus (b).

### Example 2

Container (A) contains 40 g of agent (a). Agent (a) contains (a1) 6.0 g of thiourea.

The molar mass of thiourea is 76.12 g/mol.
Agent (a) (40 g) contains 0.079 mol of thiourea.
Container (B) contains 80 g of agent (b). Agent (b) contains (b1) 10.75 g of hydrogen peroxide.
The molar mass of hydrogen peroxide is 34.02 g/mol.
Agent (b) (80 g) contains 0.316 mol of hydrogen peroxide.

To produce the ready-to-use decolorizing agent, 40 g of agent (a) is mixed together with 80 g of agent (b) (total weight of agent (a) plus (b)=120 g).

The total amount of agents (a)+(b) includes:
(a1) 6.0 g thiourea (0.079 mol in agent (a) plus (b))
(b1) 10.75 g hydrogen peroxide (0.316 mol in agent (a) plus (b))
The molar ratio of the total amount of all oxidants (b1) contained in agent (b) to the total weight of agents (a) plus (b) is 0.316 mol/0.079 mol=4.0 relative to the total weight of agents (a) plus (b).

Therefore, the oxidants (b1) are used in a molar excess of 1 to 5 times in comparison with the thiourea (a1) in the total amount of agents (a) plus (b).

The greater the excess of oxidant(s) (b1), the more complete the reaction of urea becomes. On the other hand, if the excess of oxidants (b1) is too great, the produced reaction product—i.e. the formamidine sulfinic acid, which is a very reactive reducing agent—can also decompose oxidatively. For this reason, the molar ratio of the total amount of all oxidants (b1) contained in agent (b) in relation to the total amount of thiourea (a1) contained in agent (a) is adjusted to a value of from about 1.0 to about 5.0, preferably from about 1.1 to about 4.0, more preferably from about 1.3 to about 3.0, particularly from about 1.5 to about 2.5, relative to the total weight of agents (a) plus (b).

In another particularly preferred embodiment, an inventive multi-component package unit is exemplified in that the molar ratio of the total amount of all oxidants (b1) contained in agent (b) to the total amount of thiourea (a1) contained in agent (a) has a value of from about 1.0 to about 5.0, preferably from about 1.1 to about 4.0, more preferably from about 1.3 to about 3.0 and particularly from about 1.5 to about 2.5, relative to the total weight of the agent (a).

As explained with the examples above, the molar ratio of the total amount of all oxidants (b1) contained in agent (b) to the total amount of all thiourea (a1) contained in agent (a) relative to the total weight of agents (a) plus (b) is determined by 4 factors:

(1) Amount of thiourea (a1) used in agent (a)
(2) Amount of agent (a) in container (A)
(3) Amount of oxidants (b1) used in agent (b) and
(4) Amount of agent (b) in container (B)

Depending on the desired application conditions, a person skilled in the art can adjust these 4 factors (always subject to the requirement that the molar ratio (b1)/(a1) is fulfilled).

If a very fast reaction in a concentrated medium is desired, it is advantageous to prepare a lower amount of agent (a) in the kit, wherein agent (a) contains the urea in a concentrated form—for example:

Container (A) contains 10 g of agent (a). Agent (a) contains (a1) 10.0 g of thiourea.
The molar mass of thiourea is 76.12 g/mol.
Agent (a) contains 0.131 mol of thiourea (a1).
Container (B) contains 100 g of agent (b). Agent (b) contains (b1) 11.16 g of hydrogen peroxide.
The molar mass of hydrogen peroxide is 34.02 g/mol.
Agent (b) contains 0.328 mol of hydrogen peroxide (b1).
To produce the ready-to-use decolorizing agent, 10 g of agent (a) is mixed together with 100 g of agent (b) (total weight of agent (a) plus (b)=110 g).
The total amount of agents (a)+(b) includes:
(a1) 10.0 g thiourea (0.131 mol in agent (a) plus (b))
(b1) 11.16 g hydrogen peroxide (0.328 mol in agent (a) plus (b))
The molar ratio of the total amount of all oxidants (b1) contained in agent (b) to the total weight of agents (a) plus (b) is 0.328 mol/0.131 mol=2.5 relative to the total weight of agents (a) plus (b).

On the other hand, the reaction can be better controlled when a larger amount of agent (a) is prepared in the kit, wherein agent (a) contains thiourea (a1) in a less concentrated form—for example:

Container (A) contains 40 g of agent (a). Agent (a) contains (a1) 10.0 g of thiourea.
The molar mass of thiourea is 76.12 g/mol.
Agent (a) contains 0.131 mol of thiourea (a1).
Container (B) contains 100 g of agent (b). Agent (b) contains (b1) 11.16 g of hydrogen peroxide.
The molar mass of hydrogen peroxide is 34.02 g/mol.
Agent (b) contains 0.328 mol of hydrogen peroxide (b1).
To produce the ready-to-use decolorizing agent, 40 g of agent (a) is mixed together with 100 g of agent (b) (total weight of agent (a) plus (b)=140 g).
The total amount of agents (a)+(b) includes:
(a1) 10.0 g thiourea (0.131 mol in agent (a) plus (b))
(b1) 11.16 g hydrogen peroxide (0.328 mol in agent (a) plus (b))
The molar ratio of the total amount of all oxidants (b1) contained in agent (b) to the total weight of agents (a) plus (b) is 0.328 mol/0.131 mol=2.5 relative to the total weight of agents (a) plus (b).

Containers (A) and (B) of the inventive kit-of-parts can contain agents (a) and (b) in equal or different amounts.

In a suitable embodiment, agents (a) and (b) are mixed together in a quantity ratio of 1:1 (e.g. 100 g of agent (a) and 100 g of agent (b)) (always subject to the requirement that the ratio condition (a1)/(b1) is fulfilled).

To control the reaction, it can also be advantageous that one of agent (a) or (b) is prepared in excess. Therefore, it is also advantageous that containers (A) and (B) contain agents (a) and (b) in such amounts that the quantity ratio of agent (a) to agent (b), i.e. the quantity ratio (a)/(b) has a value of from about 0.1 to about 10.0, preferably from about 0.3 to about 3.0, more preferably from about 0.5 to about 2.0 and particularly from about 0.75 to about 1.3.

### Example

Container (A) contains 10 g of agent (a).
Container (B) contains 100 g of agent (b).
The quantity ratio of agent (a) to (b), i.e. the quantity ratio (a)/(b) has a value of 10 g/100 g=0.1

### Example

Container (A) contains 20 g of agent (a).
Container (B) contains 80 g of agent (b).
The quantity ratio of agent (a) to (b), i.e. the quantity ratio (a)/(b) has a value of 20 g/80 g=0.25

### Example

Container (A) contains 50 g of agent (a).
Container (B) contains 50 g of agent (b).
The quantity ratio of agent (a) to (b), i.e. the quantity ratio (a)/(b) has a value of 50 g/50 g=1.0

Therefore, particular preference is given to a multi-component package unit (kit-of-parts) for reductive decolorization of dyed keratinous fibers exemplified in that containers (A) and (B) contain agents (a) and (b) in such amounts that the quantity ratio of agent (a) to agent (b), i.e. the quantity ratio (a)/(b) has a value of from about 0.1 to about 10.0, preferably from about 0.3 to about 3.0, more preferably from about 0.5 to about 2.0 and particularly from about 0.75 to about 1.3.

As contemplated herein, agent (a) is free from oxidants (b1).

As contemplated herein, agent (b) is free from thiourea (a1).

Multi-Component Package Unit with 3 or More Containers

Embodiments described above include two agents (a) and (b) in two separate containers. The inventive multi-component package unit can include exactly these two containers (A) and (B), which makes it a 2-component system.

However, it can be preferential in a further embodiment that the inventive multi-component package unit includes an additional container (C) or even multiple additional containers (C) and (D).

The third container (C) can be contained in the multi-component package unit if the alkalizing agent that is added for to the mixture of (a1) thiourea and (b1) oxidant for adjustment of the optimal reaction conditions should not be stored together with one of the reactants (a1) or (b1) in a container.

By packaging in three containers, the package costs associated with preparation of the containers are maximized, however, incompatibilities can be avoided in this manner and reactions taking place between the ingredients, particularly between thiourea (a1) and alkalizing agents can be avoided.

Within these additional embodiments, therefore, preference is given to a multi-component package unit exemplified in that it comprises

(III) a third container (C) containing a cosmetic agent (c).

In other words, particular preference is given to a multi-component package unit (kit-of-parts) for reductive decolorizing of keratin fibers, which is separately packaged, comprising

(I) a first container (A) containing a cosmetic agent (a) and
(II) a second container (B) containing a cosmetic agent (b) and
(III) a third container (C) containing a cosmetic agent (c),
wherein
agent (a) in the first container (A) contains
   (a1) thiourea and
agent (b) in container (B) contains
   (b1) hydrogen peroxide and
agent (c) in container (C)
   (c1) one or more alkalizing agents from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate.

If the inventive multi-component package unit comprises a third agent (c) with an alkalizing agent (c1), addition of the alkalizing agent to agent (a) is not normally mandatory. However, it can also be particularly preferred that both agent (a) and agent (c) contain one or multiple alkalizing agents.

As described above, the optimal pH value for the reaction between thiourea (a1) and oxidant(s) (b1) is in the range between about 2 to about 6.5, where from about 3.0 to about 5.5 is particularly preferred. Therefore, the mixture of agents (a) plus (b), which is the reaction mixture in which formamidine sulfinic acid is produced, a pH value is also in the range between about 2 and about 6.5.

However, the decolorizing effect of the intermediately produced formamidine sulfinic acid is now also dependent on the pH value. Ready-to-use decolorizing agents with formadine sulfinic acid have their best effect in the alkaline range. For this reason, it is particularly preferred that the agent, which is ultimately applied to the keratinous fibers for the purpose of reductive decolorization, has a pH value of from about 7.5 to about 12.5, preferably from about 8.0 to about 11.5, more preferably from about 8.5 to about 10.5 and particularly from about 8.5 to about 9.5.

Therefore, it has also been found to be particularly preferential to first adjust the mixture of agents (a) and (b) to a pH value in the range of from about 2.5 to about 6, then await the reaction to take place and then adjust the mixture of (a) and (b) with the addition of an alkalizing agent to a pH value in the range of from about 7.5 to about 12.5. For this additional increase of pH value, the mixture of agents (a) and (b) is mixed with agent (c), where agent (c) also contains one (or multiple) alkalizing agents (c1).

In this connection, particular preference is given to a multi-component package unit (kit-of-parts) for reductive decolorizing of keratin fibers, which is separately packaged, comprising

(I) a first container (A) containing a cosmetic agent (a) and
(II) a second container (B) containing a cosmetic agent (b) and
(III) a third container (C) containing a cosmetic agent (c),
wherein
agent (a) in the first container (A) contains
   (a1) thiourea and
   (a2) one or more alkalizing agents from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate, and
agent (b) in container (B) contains
   (b1) hydrogen peroxide and
agent (c) in container (C)
   (c1) one or more alkalizing agents from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate.

Additional Ingredients in Agents (a) and/or (b) and/or (c)

Agents (a), (b) and/or (c) can also contain additional ingredients and/or active ingredients. For example, the use of surfactants in agents (a), (b) and/or (c) has been found to be particularly advantageous.

The term surfactants is understood to mean amphiphilic (bifunctional) compounds having at least one hydrophobic radical and at least one hydrophilic molecule part. The hydrophobic molecule part is at least one hydrocarbon chain with from about 10 to about 30 carbon atoms. In the case of nonionic surfactants, the hydrophilic molecule part is an uncharged, highly polar structural unit.

Nonionic surfactants include, for example, at least one polyol group, a polyalkylene glycol ether group or a combination of a polyol and polyglycol ether group. Examples of such compounds include
Deposit products of from about 2 to about 50 mol ethylene oxide and/or from about 2 to about 50 mol propylene oxide on linear and branched fatty alcohols with from about 12 to about 30 carbon atoms, fatty alcohol polyglycol ether or fatty alcohol polypropylene glycol ether or mixed fatty alcohol polyethers,
Deposit products of from about 2 to about 50 mol ethylene oxide and/or from about 2 to about 50 mol propylene oxide on linear and branched fatty acids with from about 6 to about 30 carbon atoms, fatty acid polyglycol ether or fatty acid polypropylene glycol ether or mixed fatty acid polyethers,
Deposit products of from about 2 to about 50 mol ethylene oxide and/or from about 2 to about 50 mol propylene oxide on linear and branched alkyl phenols having from about 8 to about 15 carbon atoms in the alkyl group, alkyl phenolpolyglycol ethers or alkyl phenolpolypropylene ethers or mixed alkyl phenolpolyethers,
with a methyl or C₂-C₆-alkyl radical end group-closed addition products of from about 2 to about 50 moles of ethylene oxide and/or from about 0 to about 5 moles of propylene oxide on linear and branched fatty alcohols having from about 8 to about 30 carbon atoms on fatty acids having from about 8 to about 30 carbon atoms and on alkyl phenols having from about 8 to about 15 carbon atoms in the alkyl group, such as the types available under the trade names Dehydol® LS, Dehydol® LT (Cognis).
C₁₂-C₃₀ fatty acid mono- and diesters of addition products of from about 2 to about 30 moles of ethylene oxide on glycerin,
addition products of from about 5 to about 60 mol of ethylene oxide on hardened castor oil,
Polyol fatty acid esters, such as the commercially available product Hydagen® HSP (Cognis) or Sovermol®—types (Cognis),
polyalkoxylated triglycerides,
polyalkoxylated fatty acid alkylesters with the formula (Tnio-1)

R¹CO—(OCH₂CHR²)_{w}OR³ (Tnio-1)

wherein R¹CO denotes a linear branched, saturated and/or unsaturated acyl radical having from about 6 to about 22 carbon atoms, R² denotes hydrogen or methyl, R³ denotes linear or branched alkyl radicals having from about 1 to about 4 carbon atoms and w denotes numbers from
about 1 to about 20,
aminoxides,
Hydroxy mixed ethers, as described in DE-OS 197 38 866,
sorbitan fatty acid esters and addition products of ethylene oxide onto sorbitan fatty acid esters such as polysorbates,
sugar fatty acid esters and addition products of ethylene oxide on sugar fatty acid esters,
addition products of ethylene oxide on fatty acid alkanolamides and fatty amines,
sugar-based surfactants of the type of alkyl- and alkenyl oligoglycosides or
sugar-based surfactants of the type of fatty acid N-alkylpolyhydroxyalkamides.

C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and C₁₂-C₃₀ fatty acid esters have a highly polar end group (which can also be seen in the low HLB values of the compounds of this group). In the context of the present disclosure, they are considered fatty components and, therefore, are nonionic surfactants according to the definition of the present disclosure.

Furthermore, agents (a) and/or (b) can also contain one or multiple nonionic polymers.

Polymers are macromolecules having a molecular weight of at least about 1000 g/mol, preferably at least about 2500 g/mol, more preferably at least about 5000 g/mol, which includes the same, repeating organic units. Polymers are produced by polymerization of a monomer type or by polymerization of different, structurally different monomer types. If the polymer is produced by polymerization of one monomer type, it is referred to as a homopolymer. If structurally different monomer types are used in the polymerization, they are referred to as copolymers by a person skilled in the art.

The maximum molecular weight of the polymer depends on the degree of polymerization (number of polymerized monomers) and is partly determined by the polymerization method. According to the present disclosure, the maximum molecular weight of the zwitterionic polymer (d) is preferably no more than about 10⁷ g/mol, more preferably no more than about 10⁶ g/mol and even more preferably no more than about 10⁵ g/mol.

Nonionic polymers are exemplified in that they do not have any charges.

Examples of suitable nonionic polymers are vinylpyrrolidinone/vinyl acrylate copolymers, vinylpyrrolidinone, vinylpyrrolidinone/vinyl acetate copolymers, polyethylene glycols, ethylene/propylene/styrene copolymers and/or butylene/ethylene/styrene copolymers.

Moreover, the inventive agents (a) and (b) can contain additional active, auxiliary and additive ingredients, such as anionic, zwitterionic, amphoteric and/or cationic surfactants, cationic polymers such as quaternized cellulose ethers, polysiloxanes having quaternary groups, dimethyldiallylammonium chloride copolymers, acrylamide-dimethyldiallyl ammonium chloride copolymers, dimethylaminoethyl methacrylate-vinylpyrrolidinone copolymers quaternized with diethyl sulfate, vinyl pyrrolidinone-imidazolinium methochloride copolymers and quaternized polyvinyl alcohol; zwitterionic and amphoteric polymers; anionic polymers such as, for example, polyacrylic acids or crosslinked polyacrylic acids; structurants such as glucose, maleic acid and lactic acid, hair-conditioning compounds such as phospholipids, for example lecithin and kephalins; perfume oils, dimethyl isosorbide and cyclodextrins; fibre-structure-improving active ingredients, in particular mono-, di- and oligosaccharides such as, for example, glucose, galactose, fructose, fruit sugar and lactose; dyes for staining the agent; antidandruff active ingredients such as piroctone olamine, zinc omadins and climbazole; amino acids and oligopeptides; protein hydrolysates on an animal and/or vegetable basis, and in the form of their fatty acid condensation products or optionally anionically or cationically modified derivatives; vegetable oils; light stabilizers and UV blockers; active ingredients such as panthenol, pantothenic acid, pantolactone, allantoin, pyrrolidinonecarboxylic acids and their salts and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes such as fatty alcohols, beeswax, montan wax and paraffins; swelling and penetration substances such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate and PEG-3 distearate; pigments and propellants such as propane-butane mixtures, N₂O, dimethyl ether, CO₂ and air. In this context, explicit reference is made to the known monographies, e.g. Kh. Schrader, Grundlagen and Rezepturen der Kosmetika [Cosmetic principles and formulas, 2nd Edition, Hüthig Buch Verlag, Heidelberg, 1989, which reflect the corresponding knowledge of a person skilled in the art.

Decolorization of Dyed Keratinous Fibers

The inventive multi-component package unit is a system comprising agents (a) and (b) (and (c), if applicable), which is used for decolorization of previously dyed keratinous fibers, particularly human hair. The dyed keratinous fibers are usually fibers which have been colored beforehand by employing conventional oxidative dyes and/or partially oxidative dyes known to a person skilled in the art.

The decoloration agents are suitable for removing colors produced on the keratinous fibers by employing oxidizing dyes based on developer and coupler components. If the following compounds were used as developers, the colors thus produced can easily be removed effectively and almost without subsequent post-darkening by employing the decoloration agent: p-phenylendiamine, p-toluylendiamine, N,N-bis-(β-hydroxyethyl)-p-phenylendiamine, 4-N,N-bis-(β-hydroxyethyl)-amino-2-methylaniline, 2-(β-hydroxyethyl)-p-phenylendiamine, 2-(α,β-dihydroxyethyl)-p-phenylendiamine, 2-hydroxymethyl-p-phenylendiamine, bis-(2-hydroxy-5-aminophenyl)-methane, p-aminophenol, 4-amino-3-methylphenol, 2,4,5,6-tetraaminopyrimidine, 4-hydroxy-2,5,6-triaminopyrimidine, 2-hydroxy-4,5,6-triaminopyrimidine and/or 4,5-di amino-1-(β-hydroxyethyl)-pyrazol.

If the following compounds were used as couplers, the colors produced thereby can likewise be removed with very good decoloration results: m-phenylendiamine derivatives, naphthols, resorcin and resorcin derivatives, pyrazolone and m-aminophenol derivatives. Suitable coupler substances are, in particular, 1-naphthol, 1,5-, 2,7- and 1,7-dihydroxynaphthalene, 5-amino-2-methylphenol, m-aminophenol, resorcinol, resorcinol monomethyl ether, m-phenylenediamine, 1-phenyl-3-methyl-pyrazolone-5,2,4-dichloro-3-aminophenol, 1,3-bis-(2′,4′-diaminophenoxy)-propane, 2-chloro-resorcin, 4-chloro-resorcin, 2-chloro-6-methyl-3-aminophenol, 2-amino-3-hydroxypyridine, 2-methylresorcinol, 5-methylresorcinol and 2-methyl-4-chloro-5-aminophenol, 1-naphthol, 1,5-dihydroxynaphthalene, 2,7-dihydroxynaphthalene, 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcin and 2,6-dihydroxy-3,4-dimethylpyridine.

The substrate to be decolorized can also have been dyed with partially-oxidizing dyes. Nitrophenylendiamines, nitroaminophenols, azo dyes, anthraquinones or indophenols are particularly suitable partially-oxidizing dyes. The preferred partially-oxidizing dyes are the compounds known under the international designations and/or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52, as well as 1,4-diamino-2-nitrobenzol, 2-amino-4-nitrophenol, 1,4-bis-(β-hydroxyethyl)-amino-2-nitrobenzol, 3-nitro-4-(β-hydroxyethyl)-aminophenol, 2-(2′-hydroxyethyl)amino-4,6-dinitrophenol, 1-(2′-hydroxyethyl)amino-4-methyl-2-nitrobenzol, 1-amino-4-(2′-hydroxyethyl)-amino-5-chlor-2-nitrobenzol, 4-amino-3-nitrophenol, 1-(2′-ureidoethyl)amino-4-nitrobenzol, 4-amino-2-nitrodiphenylamine-2′-carboxylic acid, 6-nitro-1,2,3,4-tetrahydrochinoxaline, 2-Hydroxy-1,4-naphthochinon, pikramic acid and the salts thereof, 2-amino-6-chloro-4-nitrophenol, 4-ethylamino-3-nitrobenzoic acid and 2-chloro-6-ethylamino-1-hydroxy-4-nitrobenzol.

Moreover, the substrates to be de-colorized can also be dyed with natural dyes that occur in nature, such as Henna red, Henna neutral, Henna black, chamomile blossoms sandalwood, black tea, Cascara bark, sage, logwood, madder root, catechu, cedar and alkanna root.

The decolorizing agents as contemplated herein are designed to remove said colors and therefore themselves preferably contain no dyes, more particularly no oxidative dye precursors of the developer type and/or coupler type, as well as partially-oxidizing dyes.

In another preferred embodiment, therefore, an inventive multi-component package unit (kit-of-parts) is exemplified in that
the total amount of all dyes and oxidative dye precursors contained in agent (a) has a maximum value of about 0.2 wt. %, preferably about 0.1 wt. %, more preferably about 0.05 wt. % and particularly a maximum of about 0.01 wt. % relative to the total weight of the agent (a) and
the total amount of all dyes and oxidative dye precursors contained in agent (b) has a maximum value of about 0.2 wt. %, preferably about 0.1 wt. %, more preferably about 0.05 wt. % and particularly a maximum of about 0.01 wt. % relative to the total weight of the agent (b).

Ready-to-Use Decolorizing Agent

The ready-to-use decolorizing agent is produced by mixing the two agents (a) and (b) (and (c), if applicable) from containers (A) and (B) (and (C), if applicable), which preferably is applied after a reaction time of from about 5 to about 30 minutes to the dyed keratinous fibers or hair.

A second subject of the present disclosure, therefore, is a ready-to-use agent for reductive decolorization of dyed keratinous fibers, containing

(a1) thiourea and
(b1) one or multiple oxidizing agents from the group of hydrogen peroxide, potassium persulfate, sodium persulfate and/or ammonium persulfate.

Particular preference is given to a ready-to-use agent for reductive decolorizing of keratinous fibers containing

(a1) thiourea and
(b1) hydrogen peroxide.

Particular preference is given to a ready-to-use agent for reductive decolorizing of keratinous fibers containing

(a1) thiourea and
(b1) hydrogen peroxide and
(c1) one or more alkalizing agents from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate.

Particular preference is given to a ready-to-use agent for reductive decolorization of dyed keratinous fibers exemplified in that the molar ratio of the total amount of all oxidants (b1) contained in agent (b) to the total amount of thiourea (a1) contained in the agent has a value of from about 1.0 to about 5.0, preferably from about 1.1 to about 4.0, more preferably from about 1.3 to about 3.0 and particularly from about 1.5 to about 2.5, relative to the total weight of the agent).

The ready-to-use agent preferably contains water and has a pH value of from about 7.5 to about 12.5, preferably from about 8.0 to about 11.5, more preferably from about 8.5 to about 10.5 and particularly from about 8.5 to about 9.5.

With respect to other preferred embodiments of the ready-to-use decolorizing agent, the statements made regarding the multi-component package unit as contemplated herein apply mutatis mutandis.

Procedure

The inventive multi-component package units (kit-of-parts described above can be used in a method for reductive decolorization.

A third subject of the present disclosure is a method for reductive decolorization of keratinous fibers, including the following steps in the specified sequence.

(I) Mixture of a first agent (a) with a second agent (b), where
agent (a) is the agent disclosed in detail in the description of the first subject of the present disclosure, and
agent (b) is the agent disclosed in detail in the description of the first subject of the present disclosure, and
   (II) Mixture of the mixture of (a) and (b) produced in step (I) with a third agent (c), where
agent (c) is the agent disclosed in detail in the description of the first subject of the present disclosure, and
   (III) Application of the mixture produced in step (II) on the dyed keratinous fibers,
   (IV) Allowing the decolorizing agent to take effect,
   (V) rinsing the decolorizing agent off of the keratinous fibers.

In other words, a third subject of the present disclosure is a method for reductive decolorization of keratinous fibers, including the following steps in the specified sequence.

(I) Mixture of a first agent (a) with a second agent (b), where
agent (a) contains thiourea (a1) and
Agent (b) contains one or multiple oxidizing agents (b1) from the group of hydrogen peroxide, potassium persulfate, sodium persulfate and/or ammonium persulfate.
   (II) Mixture of the mixture of (a) and (b) produced in step (I) with a third agent (c), where
Agent (c) contains one or more alkalizing agents (c1) from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate.
   (III) Application of the mixture produced in step (II) on the dyed keratinous fibers,
   (IV) Allowing the decolorizing agent to take effect,
   (V) rinsing the decolorizing agent off of the keratinous fibers.

Particular preference is also given to a method for reductive decolorizing of dyed keratinous fibers comprising the following steps in the specified sequence.

(I) Mixture of a first agent (a) with a second agent (b), where
agent (a)
   (a1) thiourea and
   (a2) one or more alkalizing agents from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate, and
Agent (b) contains one or multiple oxidizing agents (b1) from the group of hydrogen peroxide, potassium persulfate, sodium persulfate and/or ammonium persulfate.
   (II) Mixture of the mixture of (a) and (b) produced in step (I) with a third agent (c), where
Agent (c) contains one or more alkalizing agents (c1) from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate.
   (III) Application of the mixture produced in step (II) on the dyed keratinous fibers,
   (IV) Allowing the decolorizing agent to take effect,
   (V) rinsing the decolorizing agent off of the keratinous fibers.

In step (I) the decolorizing agent is produced by mixing agents (a) and (b). Agents (a) and (b) are the two agents of the inventive multi-component package unit. With respect to other preferred embodiments of agents (a) and (b), the statements made regarding the multi-component package unit as contemplated herein apply mutatis mutandis.

After mixture of agents (a) and (b), the reaction between thiourea (a1) and oxidant (b1) is initiated. In order to guarantee the most complete reaction possible, it is advantageous to wait with step (II) of the method for from about 5 to about 60 minutes, preferably from about 10 to about 50 minutes, more preferably from about 15 to about 45 minutes and particularly from about 20 to about 35 minutes.

In a further preferred embodiment, a method as contemplated herein is exemplified in that a period of from about 5 to about 60 minutes, preferably from about 10 to about 50 minutes, more preferably from about 15 to about 45 minutes and particularly from about 20 to about 35 minutes passes between steps (I) and (II).

In step (II), by mixing the mixture of agents (a) and (b) produced in step (I) with a third agent (c), the ready-to-use decolorizing agent that has the optimal pH value for the reductive color removal is produced.

Then this ready-to-use decolorizing agent can be applied in step (III) to the keratinous fibers or hair, left to take effect in step (IV) and washed out in step (V).

In step (IV), the decolorizing agent is left on the keratinous fiber to take effect for from about 5 to about 80 minutes, preferably from about 15 to about 70 minutes and particularly from about 20 to about 65 minutes.

With respect to other preferred embodiments of the inventive method, the statements made regarding the multi-component package unit and the ready-to-use agent as contemplated herein apply mutatis mutandis.

### Examples

### 1.1. Coloration

The following formulations were produced (all data in wt. %):

Dye Cream (F1)

| Raw material | wt. % |
|---|---|
| Cetearyl alcohol | 8.5 |
| C12-C18 fatty alcohols | 3.0 |
| Ceteareth-20 | 0.5 |
| Ceteareth-12 | 0.5 |
| Plantacare 1200 UP (laurylglucoside, 50-53% | 2.0 |
| hydrous solution) | |
| Sodium laureth-6 carboxylate (21% i | 10.0 |
| hydrous solution) | |
| Sodium myreth sulfate (68-73% hydrous solution) | 2.8 |
| Sodium acrylate, trimethylammoniopropyl- | 3.8 |
| acrylamide chloride | |
| copolymer (19-21% hydrous solution) | |
| Potassium hydroxide | 0.83 |
| p-toluylendiamine, sulfate | 2.25 |
| m-aminophenol | 0.075 |
| 2-amino-3-methylphenol | 0.12 |
| Resorcin | 0.62 |
| 4-chlorresorcin | 0.26 |
| 3-amino-2-methylamino-6-methoxypyridin | 0.04 |
| 1,3-bis(2,4-diaminophenoxy)propane, | 0.05 |
| tetrahydrochloride | |
| Ammonium sulfate | 0.1 |
| Sodium sulfate | 0.4 |
| Ascorbic acid | 0.1 |
| 1-Hydroxyethane-1,1-diphosphonic acid | 0.2 |
| (60% hydrous solution) | |
| Ammonia (25% hydrous solution) | 7.2 |
| Water | Ad 100 |

Oxidant (Ox)

| Raw material | wt. % |
|---|---|
| Sodium benzoate | 0.04 |
| Dipicolinic acid | 0.1 |
| Di-sodium pyrophosphate | 0.1 |
| Potassium hydroxide | 0.09 |
| 1,2-Propylenglycol | 1.0 |
| 1-Hydroxyethane-1,1-diphosphonic acid | 0.25 |
| (60% hydrous solution) | |
| Paraffinum liquidum | 0.30 |
| Steartrimonium chloride | 0.39 |
| Cetearyl alcohol | 3.4 |
| Ceteareth-20 | 1.0 |
| Hydrogen peroxide (50% hydrous | 12.0 |
| solution) | |

The dye cream (F1) and the oxidant (Ox) were then mixed together in a ratio of 1:1 and applied to the hair strands (Kerling medium blond, 9-0). The weight ratio of application mixture to hair was 4:1, exposure time 30 minutes at a temperature of 32 degrees Celsius. The strands were then rinsed with water, dried and left to rest at room temperature for at least 24 hours. The strands were dyed in a dark-brown color.

### 2. Decolorization

(1) 7.5 g of thiourea (0.0985 mol) were dissolved in 100 ml of water for production of agent
(a). With addition of monoethanolamine, this solution is brought to a pH value of 5.0.
(2) 6.7 g of hydrogen peroxide (0.197 mol) were dissolved in 100 ml of water for production of agent (b).
(3) Then 100 g of agent (a) were mixed with 100 g of agent (b). The pH value of this mixture was in the range of 4.5 to 5.5.
(4) This mixture was left to take effect for 20 minutes at room temperature and shaken occasionally during that time.
(5) The pH value of the mixture obtained in step (4) was adjusted to a value of 8.0 with addition of a hydrous ethanolamine solution (agent (c)).
(6) The ready-to-use decolorizing agent produced in step (5) was applied to the dyed hair strands, left there to take effect for a period of 60 minutes at room temperature and then rinsed out with water. Then the hair was dried.

### 3. Evaluation of the Decolorizing Effect

The hair strands were measured by colorimetry before dying, after dyeing and after decolorization (measurement of lab values). The L-value was used as a measure for the lightening effect of the agent. The L-value specifies the lightness of a coloring (L=1000=diffuse white; L=0=black). The greater the L-value, the lighter the coloring and the better the decolorizing result.

| | | ΔL-value versus |
|---|---|---|
| | L-value | uncolored |
| Kerling 9-0 medium-blond, undyed | 42.20 | — |
| Kerling 9-0 medium-blond, dyed | 20.89 | 21.31 |
| Kerling 9-0, medium-blond, dyed and | 27.14 | 15.06 |
| decolorized | | |

Significant decolorization was observed after application of the decolorizing agent.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Multi-component package unit (kit-of-parts) for reductive decolorizing of keratin fibers comprising, separately packaged,
(I) a first container (A) comprising a cosmetic agent (a) and
(II) a second container (B) comprising a cosmetic agent (b),
wherein
agent (a) in the first container (A) comprises
(a1) thiourea and
agent (b) in container (B) comprises
(b1) one or multiple oxidizing agents chosen from the group of hydrogen peroxide, potassium persulfate, sodium persulfate and/or ammonium persulfate.

2. Multi-component package unit according to claim 1 wherein agent (a) in container (A) comprises—relative to the total weight of agent (a)—from about 1.0 to about 80.0 wt. % thiourea.

3. Multi-component package unit according to claim 1, wherein
agent (a) in container (A) further comprises
(a2) one or more alkalizing agents from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate.

4. Multi-component package unit according to claim 1, wherein
agent (b) in container (B) comprises
(b1) hydrogen peroxide.

5. Multi-component package unit according to claim 1 wherein agent (b) in the second container (B) comprises from about 0.1 to about 12.0 wt. % hydrogen peroxide relative to the total weight of agent (b).

6. Multi-component package unit according to claim 1 wherein the molar ratio of the total amount of all oxidants (b1) included in agent (b) to the total amount of thiourea (a1) included in agent (a) has a value of from about 1.0 to about 5.0, relative to the total weight of the agent (a).

7. Multi-component package unit according to claim 1 wherein containers (A) and (B) comprise agents (a) and (b), respectively, in such amounts that the quantity ratio of agent (a) to agent (b) has a value of from about 0.1 to about 10.0.

8. Multi-component package unit according to claim 1, further comprising
(III) a third container (C) comprising a cosmetic agent (c).

9. Multi-component package unit according to claim 8 wherein
agent (c) in container (C) comprises
(c1) one or more alkalizing agents chosen from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate.

10. Ready-to-use agent for reductive decolorizing of dyed keratinous fibers comprising
(a1) thiourea and
(b1) one or multiple oxidizing agents chosen from the group of hydrogen peroxide, potassium persulfate, sodium persulfate and/or ammonium persulfate.

11. Agent according to claim 10 further comprising
(c1) one or more alkalizing agents from the group comprising ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate.

12. Agent according to claim 10 wherein the molar ratio of the total amount of all oxidants (b1) included in agent (b) to the total amount of thiourea (a1) included in agent (a) has a value of from about 1.0 to about 5.0, relative to the total weight of the agent (a).

13. Method for reductive decolorizing of dyed keratinous fibers comprising the following steps in the specified sequence:
(I) Mixing a first agent (a) with a second agent (b), where
agent (a) comprises (a1) thiourea, and
agent (b) comprises (b1) one or multiple oxidizing agents chosen from the group of hydrogen peroxide, potassium persulfate, sodium persulfate and/or ammonium persulfate.
(II) Mixing the mixture of (a) and (b) produced in step (I) with a third agent (c), where
agent (c) comprises a cosmetic agent (c).
(III) Applying the mixture produced in step (II) on the dyed keratinous fibers,
(IV) Allowing the decolorizing agent to take effect,
(V) Rinsing the decolorizing agent off of the keratinous fibers.

14. Method according to claim 13 wherein a period of from about 5 to about 60 minutes passes between steps (I) and (II).

15. Multi-component package unit according to claim 1 wherein agent (a) in container (A) comprises—relative to the total weight of agent (a)—from about 7.0 to about 20.0 wt. % thiourea.

16. Multi-component package unit according to claim 1 wherein agent (b) in the second container (B) comprises from about 4.0 to about 8.0 wt. % hydrogen peroxide relative to the total weight of agent (b).

17. Multi-component package unit according to claim 1 wherein the molar ratio of the total amount of all oxidants (b1) included in agent (b) to the total amount of thiourea (a1) included in agent (a) has a value of from about 1.5 to about 2.5, relative to the total weight of the agent (a).

18. Multi-component package unit according to claim 1 wherein containers (A) and (B) contain agents (a) and (b) in such amounts that the quantity ratio of agent (a) to agent (b) has a value of from about 0.75 to about 1.3.

19. Multi-component package unit according to claim 1, wherein:
agent (a) in container (A) comprises—relative to the total weight of agent (a)—from about 1.0 to about 80.0 wt. %;
agent (a) in container (A) further comprises
(a2) one or more alkalizing agents from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate;
agent (b) in the second container (B) comprises from about 0.1 to about 12.0 wt. % hydrogen peroxide relative to the total weight of agent (b);
the molar ratio of the total amount of all oxidants (b1) included in agent (b) to the total amount of thiourea (a1) included in agent (a) has a value of from about 1.0 to about 5.0, relative to the total weight of the agent (a); and
containers (A) and (B) comprise agents (a) and (b), respectively, in such amounts that the quantity ratio of agent (a) to agent (b) has a value of from about 0.1 to about 10.0.

20. Multi-component package unit according to claim 1, wherein:
agent (a) in container (A) comprises—relative to the total weight of agent (a)—from about 1.0 to about 80.0 wt. % thiourea;
agent (a) in container (A) further comprises
(a2) one or more alkalizing agents from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate
the multi-component package unit further comprises (III) a third container (C) comprising a cosmetic agent (c); and
agent (c) in container (C) comprises
(c1) one or more alkalizing agents chosen from the group of ammonia, 2-aminoethan-1-ol, 2-amino-2-methylpropane-1-ol, arginine, lysine, ornithine, histidine, potassium hydroxide, sodium hydroxide, magnesium hydroxide, calcium hydroxide, sodium metasilicate, potassium metasilicate, sodium carbonate, potassium carbonate, sodium bicarbonate and/or potassium bicarbonate.
